(19) United States
(12) Patent Application Publication
(10) Pub. No.: US 2009/0306569 A1
(43) Pub. Date: Dec. 10, 2009

(54) TIP BANDAGE

(76) Inventor: Carolyn F. Cook, Birmingham, AL (US)

Correspondence Address: MICHAEL RIES, 318 PARKER PLACE, OSWEGO, IL, 60543, US

(21) Appl. No.: 12/134,605

(22) Filed: Jun. 6, 2008

(51) Int. Cl. A61F 13/10; A61F 13/02

(52) U.S. Cl. 602/43; 602/54

(57) ABSTRACT

Disclosed is a bandage for healing wounds on a digit of a user. The bandage includes an elongated sleeve and a strip. The elongated sleeve is configured to be received on the digit of the user. The elongated sleeve includes an open end portion and a closed end portion. The closed end portion of the elongated sleeve is received on a tip portion of the digit of the user when the elongated sleeve is received thereon. An inner surface of the elongated sleeve is disposed with a medicament for healing wounds on the digit of the user. The strip is configured on a peripheral edge portion of the open end portion of the elongated sleeve. The strip is adapted to secure the elongated sleeve to the digit of the user for enabling a comfortable fit of the elongated sleeve thereon.

### FIELD OF THE INVENTION

The present invention relates generally to bandages for covering and protecting wounds, and, more particularly, to a bandage for healing wounds on a digit of a user and a tip portion thereof.

### BACKGROUND OF THE INVENTION

Bandages are configured to cover and protect wounds. Generally, the bandages are flat and rectangular in shape. Further, a bandage is configured with an absorbent pad on a surface thereof such that the absorbent pad is received on a wound of a user when the bandage is used to cover the wound. The absorbent pad is medicated with an antiseptic agent to heal the wound of the user. Accordingly, the bandage protects the wound against dirt and other contaminants that may cause further infection. Since the bandage provides a proper protection of the wound from further infection, the bandage facilitates faster healing of the wound.

However, the conventional bandage is useful for covering and protecting relatively flat body parts of a user and is not suited for use on body parts having curved contours such as a tip portion of a digit, for example a finger of a hand. The conventional bandage tends to wrinkle and crimp when applied on the tip portion of the digit. Accordingly, the absorbent pad is partially received on the wound present on the tip portion of the digit. Further, the conventional bandage is unable to adjust to the curved contours of the tip portion of the digit and accordingly tends to protrude away from skin of the tip portion of the digit, thereby creating an opening for introduction of dust, dirt, bacteria and the like. More specifically, the conventional bandage is unable to cover and protect a wound on a tip portion of a digit.

Accordingly, based on the foregoing, there is a need for a bandage, which is capable of being comfortably received on a digit of a user for covering a wound. More specifically, there is a need for a bandage, which is capable of protecting and healing wounds on a digit of a user and a tip portion thereof. Moreover, there is a need for a bandage which enables quick healing of wounds.

### SUMMARY OF THE INVENTION

In view of the foregoing disadvantages inherent in the prior art, the general purpose of the present invention is to provide a bandage for healing wounds on a digit of a user that is configured to include all the advantages of the prior art, and to overcome the drawbacks inherent therein.

Accordingly, an object of the present invention is to provide a bandage for healing wounds on a digit of a user, which is configured to be comfortably received on the digit of the user for healing wounds thereon.

Another object of the present invention is to provide a bandage for healing wounds on a digit of a user, which is capable of providing a sterile covering on the wounds, thereby enabling quick healing of the wounds.

In light of the above objects, in one aspect of the present invention, disclosed is a bandage for healing wounds on a digit of a user. The bandage includes an elongated sleeve and a strip. The elongated sleeve includes an open end portion and a closed end portion. The elongated sleeve is configured to be received on the digit of the user. More specifically, the closed end portion of the elongated sleeve is received on a tip portion of the digit of the user when the elongated sleeve is received on the digit of the user. Moreover, an inner surface of the elongated sleeve is disposed with a medicament for healing the wounds on the digit of the user. The strip is configured on a peripheral edge portion of the open end portion of the elongated sleeve. The strip is adapted to secure the elongated sleeve to the digit of the user for enabling a comfortable fit of the elongated sleeve thereon.

In another aspect of the present invention, the strip includes an adhesive strip configured thereon for enabling the elongated sleeve to be secured to the digit of the user.

In yet another aspect of the present invention, the elongated sleeve and the strip is composed of a waterproof material.

In still another aspect of the present invention, the elongated sleeve is composed of a transparent material.

In still another aspect of the present invention, the elongated sleeve and the strip is composed of a flexible material.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, in which:

FIG. 1 illustrates a perspective view of a bandage for healing wounds on a digit of a user, in accordance with an embodiment of the present invention; and

FIG. 2 illustrates a top view of the bandage shown in FIG. 1.

Like reference numerals refer to like parts throughout the description of several views of the drawings.

### DETAILED DESCRIPTION OF THE INVENTION

The exemplary embodiments described herein detail for illustrative purposes are subject to many variations in structure and design. It should be emphasized, however, that the present invention is not limited to a particular bandage for healing wounds on digit of user, as shown and described. It is understood that various omissions and substitutions of equivalents are contemplated as circumstances may suggest or render expedient, but these are intended to cover the application or implementation without departing from the spirit or scope of the claims of the present invention.

The terms “a” and “an” herein do not denote a limitation of quantity, but rather denote the presence of at least one of the referenced item.

The present invention provides a bandage for healing wounds. More specifically, the bandage is adapted to heal wounds on a digit of a user. The term “wounds” as used herein, refers to various types of bodily injuries such as a cut, an abrasion, a bruise, a blister, a hangnail, a paper cut, a burn, a skin puncture and the like. The term “digit” as used herein, refers to a finger or a thumb of a hand or a toe. The bandage of the present invention is adapted to be comfortably received on the digit of the user. The bandage may be made from a waterproof material to prevent the wound to be exposed to water.

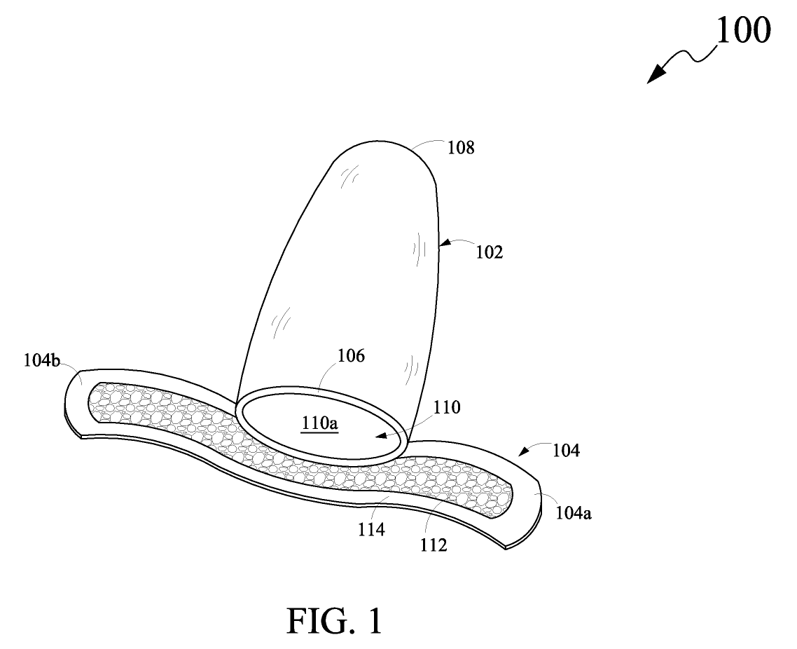

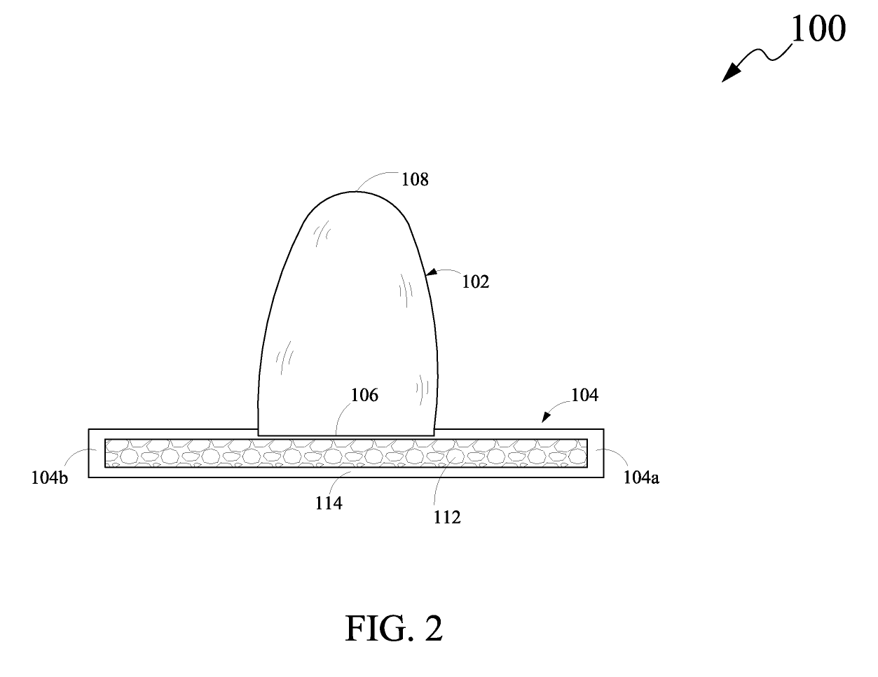

Referring to FIG. 1 and FIG. 2, a bandage 100 for healing wounds on a digit of a user is illustrated. More specifically, FIG. 1 illustrates a perspective view of the bandage 100, in accordance with an embodiment of the present invention and FIG. 2 illustrates a top view of the bandage 100.

As shown in FIGS. 1 and 2, the bandage 100 includes an elongated sleeve 102 and a strip 104 configured on the elongated sleeve 102. The elongated sleeve 102 includes an open end portion 106, a closed end portion 108 and a cavity 110 having an inner surface 110a. In one embodiment of the present invention, the open end portion 106 may be circular in shape. The cavity 110 extends between the open end portion 106 and the closed end portion 108 of the elongated sleeve 102. The elongated sleeve 102 is configured to be received on a digit of a user (not shown) in a manner such that the cavity 110 receives a portion of the digit therein. The elongated sleeve 102 may be composed of various water proof materials such as, but not limited polypropylene. Further, the elongated sleeve 102 may be composed of a flexible material, such as, but not limited to a woven fabric incorporating elastic threads. Further the elongated sleeve 102 may also be composed of a transparent and light weight material such as but not limited to plastic. Moreover, the elongated sleeve 102 may be composed of various synthetic resin materials.

In use, the user may insert the portion of the digit in the cavity 110 through the open end portion 106 of the elongated sleeve 102. Thereafter, the user may move the digit further into the cavity 110 such that a tip portion of the digit is in contact with the closed end portion 108 of the elongated sleeve 102 and the inner surface 110a of the cavity 110 is in contact with a substantial portion of the digit of the user.

The inner surface 110a of the elongated sleeve 102 is disposed with a medicament for healing the wounds on the digit of the user. The medicament may include a protective lotion, a salve, an ointment, an antiseptic agent and the like. The medicament is adapted to configure a sterile covering over the wounds on the digit of the user, when the elongated sleeve 102 is received thereon, thereby preventing further infection of the wounds by foreign particles such as dust, dirt, bacteria and the like. In one embodiment of the present invention, a pad may be configured on the inner surface 110a of the elongated sleeve 102 for providing cushioning over the wounds. The pad may be disposed with the medicaments for healing the wounds.

The bandage 100 may be secured to the digit of the user with the help of the strip 104 having end portions 104a and 104b. The strip 104 is configured on a peripheral edge portion of the open end portion 106 of the elongated sleeve 102, as shown in FIG. 1. In one embodiment of the present invention, the strip 104 may be separately attached to the peripheral edge portion of the open end portion 106 by conventional coupling means such as adhesives, weaving, sewing and the like. In another embodiment of the present invention, the elongated sleeve 102 and the strip 104 may be configured as a single unit such that the strip 104 extends from the elongated sleeve 102.

The strip 104 is adapted to secure the elongated sleeve 102 to the digit of the user when the elongated sleeve 102 is received thereon for enabling a comfortable fit of the elongated sleeve 102 on the digit of the user. In one embodiment of the present invention, the strip 104 includes an adhesive strip 112 configured on a surface 114 of the strip 104 and extending between the end portions 104a and 104b of the strip 104. The adhesive strip 112 is adapted to releasably secure the elongated sleeve 102 to the digit of the user. More specifically, after the elongated sleeve 102 is received on the digit of the user, the strip 104 may be wrapped around the digit such that the adhesive layer 112 adheres to a surface of the digit.

In one embodiment of the present invention, the strip 104 may have a suitable length such that the end portions 104a and 104b overlap with each other when the strip 104 is wound over the digit. In another embodiment of the present invention, the adhesive strip 112 may be covered with a release liner (not shown) for maintaining adhesive property of the adhesive strip 112. The release liner is required to be removed once the elongated sleeve 102 is received on the digit of the user, allowing the adhesive strip 112 to adhere to a surface of the digit. In yet another embodiment of the present invention, a suitable coupling means, such as a clip assembly, may be configured at the end portions 104a and 104b of the strip 104 for securing the elongated sleeve 102 to the digit of the user. Moreover, the strip 104 may be composed of a water proof material such as but not limited to propylene, plastic and the like. It will be however, evident to a person skilled in the art that the strip 104 may be composed of any other suitable material known in the art.

The above-described presently preferred embodiment can be modified into many alternative preferred embodiments. In one embodiment of the present invention, length of the elongated sleeve 102 of the bandage 100 may be varied for enabling the elongated sleeve 102 to be received on various portions of the digit. For example, the elongated sleeve 102 may be configured to have a length such that the elongated sleeve 102 extends up to a first joint of the digit. Similarly, the elongated sleeve 102 may be configured to have a length to extend up to a second joint, or to a knuckle of the digit. In one exemplary embodiment of the present invention, the length of the elongated sleeve 102 may range from about half an inch to about one and half inches. In another embodiment of the present invention, the elongated sleeve 102 may be configured in a variety of colors and textures to provide customized aesthetic appeal. Moreover, in yet another embodiment of the present invention, the elongated sleeve 102 of the bandage 100 may be configured to include a design representing one or more cartoon characters.

Further, in still another embodiment of the present invention, the open end portion 106 of the bandage 100 may be configured in a multitude of sizes having various diameters to accommodate digits of different sizes.

Various embodiments of the present invention offer following advantages. The bandage, as described herein, is capable of being received on a digit of a user for healing wounds present on the digit. Further, the bandage is configured to be capable of being easily received and secured on the digit of a user and more specifically on a tip portion of the digit. Furthermore, the bandage is capable of providing a sterile covering on the wounds on the digit and/or the tip portion of the digit. Moreover, the flexible nature of the bandage allows the encased digit to flex and bend with minimum resistance.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, and to thereby enable others skilled in the art to best utilize the present invention and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the spirit or scope of the claims of the present invention.

What is claimed is:

1. A bandage for healing wounds on a digit of a user, the bandage comprising:
an elongated sleeve configured to be received on the digit of the user, the elongated sleeve comprising an open end portion and a closed end portion, the closed end portion of the elongated sleeve adapted to be received on a tip portion of the digit of the user when the elongated sleeve is received thereon, wherein an inner surface of the elongated sleeve is disposed with a medicament for healing the wounds on the digit of the user; and
a strip configured on a peripheral edge portion of the open end portion of the elongated sleeve, the strip adapted to secure the elongated sleeve to the digit of the user for enabling a comfortable fit of the elongated sleeve thereon.

2. The bandage of claim 1, wherein the strip comprises an adhesive strip configured thereon for enabling the elongated sleeve to be secured to the digit of the user.

3. The bandage of claim 1, wherein the strip is composed of a water proof material.

4. The bandage of claim 1, wherein the elongated sleeve is composed of a water proof material.

5. The bandage of claim 1, wherein the elongated sleeve is composed of a transparent material.

6. The bandage of claim 1, wherein the elongated sleeve is composed of a flexible material.
